(19) United States
(12) Patent Application Publication
(10) Pub. No.: US 2024/0090909 A1
(43) Pub. Date: Mar. 21, 2024

(54) CLOT RETRIEVAL SYSTEM WITH EXPANDABLE CLOT ENGAGING FRAMEWORK

(71) Applicant: Neuravi Limited, Galway (IE)

(72) Inventor: Stephen WHELAN, Galway (IE)

(73) Assignee: Neuravi Limited, Galway (IE)

(21) Appl. No.: 18/522,495

(22) Filed: Nov. 29, 2023

Related U.S. Application Data
(62) Division of application No. 16/908,161, filed on Jun. 22, 2020.

(51) Int. Cl. A61B 17/221

(52) U.S. Cl. CPC A61B 17/221; A61B 2017/22001

(57) ABSTRACT

A clot removal device for removing a clot from a body vessel is presented. The device can facilitate clot retrieval by expanding in such a way as to engage the clot over a significant surface area. The clot can have at least one firm portion and at least one soft portion. At least one firm portion of the clot can be pinched by a proximal portion of the device while at least one soft portion of the clot can be retained by the distal portion of the device upon extraction. However, dislodgement and removal of the clot can cause softer portions the clot to shear or break, resulting in small floating fragments of clot in the vessel no longer attached to the main clot. This device can also capture and retain sheared or expanded distal portions of the clot upon removal of the clot from a vessel.

### CROSS REFERENCE TO RELATED APPLICATION

The present application is a divisional application of U.S. patent application Ser. No. 16/908,161 filed Jun. 22, 2020. The entire contents of which are hereby incorporated by reference.

### FIELD

The present disclosure generally relates to devices and methods for removing blockages from blood vessels during intravascular medical treatments.

### BACKGROUND

This invention relates to devices intended for removing acute blockages from blood vessels. Acute obstructions may include clots, misplaced devices, migrated devices, large emboli and the like. Thromboembolism occurs when part or all of a thrombus breaks away from the blood vessel wall. This clot (now called an embolus) is then carried in the direction of blood flow. An ischemic stroke may result if the clot originates in the venous system or is the right side of the heart and lodges in a pulmonary artery or branch thereof. Clots may also develop and block vessels locally without being released in the form of an embolus—this mechanism is common in the formation of coronary blockages.

Clots can often be subject to shearing or breakage upon removal from a vessel. The invention is particularly suited to removing clots in their entirety, without shearing off and/or leaving behind residual portions of the clot. The device can be used in cerebral arteries in patients suffering acute ischemic stroke (AIS), coronary native or graft vessels in patients suffering from myocardial infarction (MI), and in pulmonary arteries in patients suffering from pulmonary embolism (PE) and other peripheral arterial and venous vessels in which the clot is causing an occlusion.

### SUMMARY

A clot removal device for removing a clot from a body vessel is presented herein. The device can facilitate clot retrieval by expanding in such a way as to engage the clot over a significant surface area. The clot can have at least one firm portion and at least one soft portion. At least one firm portion of the clot can be pinched by a proximal portion of the device while at least one soft portion of the clot can be retained by the distal portion of the device upon extraction. The pinch can be achieved by forwarding a microcatheter or intermediate catheter over the device until a portion of the clot is compressed between the tip of the catheter and a crown or strut on the device. However, dislodgement and removal of the clot can cause softer portions the clot to shear or break, resulting in small floating fragments of clot in the vessel no longer attached to the main clot. This device is also intended to capture or retain sheared or expanded distal portions of the clot upon removal of the clot from a vessel.

In some examples presented herein, a microcatheter and a guidewire can be inserted into a patient's vasculature via a retrieval catheter and advanced across a clot. When the microcatheter is in position, the guidewire can be removed to allow a clot retrieval device to be advanced through the microcatheter to the clot. The device can be advanced in a collapsed configuration. Then, the microcatheter can be retracted while the position of the device is maintained to deploy the device across the clot.

In some examples, the device can include a clot engagement framework, a hypotube, and a distal emboli protection system. The engagement framework can have a collapsed delivery configuration, a clot-engaging deployed configuration, and a clot pinching configuration. The engagement framework can also have a proximal end, a distal end, and a distal tip. The hypotube can include a distal end at least partially surrounded by the clot engagement framework in a spiral configuration. The distal emboli protection system can expand from a collapsed delivery configuration within the hypotube to a deployed configuration distal to the hypotube's distal end.

In some examples, after the device is deployed across a clot, the microcatheter can be advanced distally into the engagement framework to move the engagement framework to the clot-pinching configuration. In the clot-pinching configuration, the struts of the proximal end of the engagement framework can be compressed to pinch a clot. Then, the distal emboli protection system can be advanced through the hypotube in a collapsed delivery configuration and deployed to an expanded configuration distally to the distal end of the hypotube. The struts of the engagement framework in the deployed configuration can have a clot gripping surface to engage and hold onto a firm portion of the clot. Once the device is in the pinching configuration and the distal emboli protection system has been deployed, the microcatheter and device can be retracted towards a retrieval catheter. During retraction, it is possible that small, softer portions of the clot might break or shear apart from the firmer, larger clot mass. The distal emboli protection system can serve as a catch-all mechanism to retain these softer portions of the clot upon removal of the device. The device can be removed through the retrieval catheter.

In some examples described herein, the device can include a pull wire and a stentriever. The pull wire can be affixed approximate a distal end of the stentriever. The stentriever can include a proximal spiral section, a distal cylindrical body section, and a distal cone. At least a portion of the spiral section can encircle the pull wire. The stentriever can expand from a collapsed delivery configuration to a pinching configuration upon retraction of a microcatheter as previously described, and then further expand to an expanded configuration. In the pinching configuration, the distal cylindrical body section can have a first diameter, and the microcatheter can be advanced distally towards the proximal spiral section to collapse the openings of the proximal spiral portion to pinch a firm portion of a clot between the microcatheter's distal end and the proximal spiral section. Once the stentriever is in the pinching configuration, the pull wire can be retracted to expand the stentriever to the expanded configuration. In the expanded configuration, the distal cylindrical body section can expand radially to a second, greater diameter and engage any soft portions of the clot that might otherwise be vulnerable to shearing or breakage upon removal of the device. After the stentriever is in the expanded configuration, the microcatheter and device can be retracted through the vasculature into a retrieval catheter for removal from the patient.

An example method for treating a patient with an occluded vessel can include one or more of the following steps presented in no particular order, and the method can include additional steps not included here. A clot engagement framework positioned within a microcatheter can be delivered to a target occluded vessel. A hypotube with a distal emboli protection system can be delivered to the occluded vessel with the clot engagement framework. The clot engagement framework can be deployed to contact at least a portion of the clot. The hypotube can be advanced across the clot. The microcatheter can be advanced over a proximal portion of the clot engagement framework thereby pinching the clot. The distal emboli protection system can be deployed. Then, the clot engagement framework, the distal emboli protection system and clot can be withdrawn from the patient. The method can further include gripping the clot with an outer surface of the hypotube. The method can also include capturing clot fragments with the distal emboli protection system.

Another example method for treating a patient with an occluded vessel can include one or more of the following steps presented in no particular order, and the method can include additional steps not included here. A device comprising a stentriever and pull wire can be delivered to the occluded vessel through a microcatheter. The stentriever can be deployed to contact at least a portion of the clot such that a proximal spiral portion of the stentriever forms a spiral and a distal cylindrical portion of the stentriever forms a cylindrical body. At least a portion of the clot can be pinched with the spiral portion of the stentriever. The pull wire can be retracted to radially expand the cylindrical portion of the stentriever. The microcatheter and the device can be withdrawn in unison from the vessel. The device, microcatheter, and clot can then be removed from the patient. The method can further include collecting at least a portion of the clot within a distal cone of the stentriever. The method can also comprise positioning the device such that at least a portion of the spiral section encircles the pull wire.

### BRIEF DESCRIPTION OF THE DRAWINGS

The invention will be more clearly understood from the following description of some embodiments thereof, given by way of example only, with reference to the accompanying drawings, in which:

FIGs. 1A to 1B show the delivery of a clot retrieval device to a targeted location within a vessel according to aspects of the present invention;

FIGS. 2A to 2C show various mechanical components of a clot retrieval device according to aspects of the present invention;

FIGS. 3A to 3E show removal of a clot from a vessel using a clot retrieval device according to aspects of the present invention;

FIGS. 4A to 4B show the delivery of a clot retrieval device to a targeted location within a vessel according to aspects of the present invention;

FIGS. 5A and 5B show various mechanical components of a clot retrieval device according to aspects of the present invention;

FIGS. 6A to 6C show removal of a clot from a vessel using a clot retrieval device according to aspects of the present invention;

FIG. 7 is a flow diagram depicting a method of treating a patient with an occluded vessel according to aspects of the present invention; and

FIG. 8 is a flow diagram depicting a method of treating a patient with an occluded vessel according to aspects of the present invention.

### DETAILED DESCRIPTION

Specific embodiments of the present invention are now described in detail with reference to the Figures, wherein identical reference numbers indicate identical or functionally similar elements. The terms “distal” and “proximal” are used in the following description with respect to a position or direction relative to the treating physician. “Distal” or “distally” are a position distant from or in a direction away from the physician. “Proximal” or “proximally” or “proximate” are a position near or in a direction toward the physician.

Accessing cerebral, coronary, and pulmonary vessels involves the use of a number of commercially available products and conventional procedural steps. Access products such as guidewires, guide catheters, angiographic catheters and microcatheters are described elsewhere and are regularly used in catheter lab procedures. It is assumed in the descriptions below that these products and methods are employed in conjunction with the device and methods of this invention and do not need to be described in detail.

The following detailed description is merely explanatory in nature and is not intended to limit the invention or the application and uses of the invention. Although the description of the invention is in many cases in the context of treatment of intercranial arteries, the invention may also be used in other body passageways as previously described.

The expandable members of the designs disclosed are desirably made from a material capable of recovering its shape automatically once released from a highly strained delivery configuration. A superelastic material such as Nitinol or an alloy of similar properties is particularly suitable. The material could be in many forms such as wire or strip or sheet or tube. A particularly suitable manufacturing process is to laser cut a Nitinol tube and then heat set and electropolish the resultant structure to create a framework of struts and connecting elements. This framework can be any of a huge range of shapes as disclosed herein and may be rendered visible under fluoroscopy through the addition of alloying elements (such as platinum) or through a variety of other coatings or marker bands.

When used herein, the terms “tubular” and “tube” are to be construed broadly and are not limited to a structure that is a right cylinder or strictly circumferential in cross-section or of a uniform cross-section throughout its length. For example, the tubular structure or system is generally illustrated as a substantially right cylindrical structure. However, the tubular system may have a tapered or curved outer surface without departing from the scope of the present invention.

This device is intended to facilitate clot retrieval by expanding in such a way as to engage the clot over a significant surface area. A portion of the clot can be pinched between the tip of the catheter and the nitinol struts of the device. The clot can have at least one firm portion and at least one soft portion. At least one firm portion of the clot can be pinched by proximal portion of the device while at least one soft portion of the clot can be retained by the distal portion of the device upon extraction. The pinch can be achieved by forwarding a microcatheter or intermediate catheter over the device until a portion of the clot is compressed between the tip of the catheter and a crown or strut on the device. This pinch facilitates removal of the clot as it increases the grip of the device on the clot, particularly fibrin rich clots. It may also elongate the clot reducing the dislodgement force by pulling the clot away from the vessel wall during the dislodgement process. However, dislodgement and removal of the clot can cause soft portions of the clot to shear or break, resulting in small floating fragments of clot in the vessel no longer attached to the main clot. This device is also intended to capture and retain soft, sheared, or expanded distal portions of the clot upon removal of the clot from a vessel.

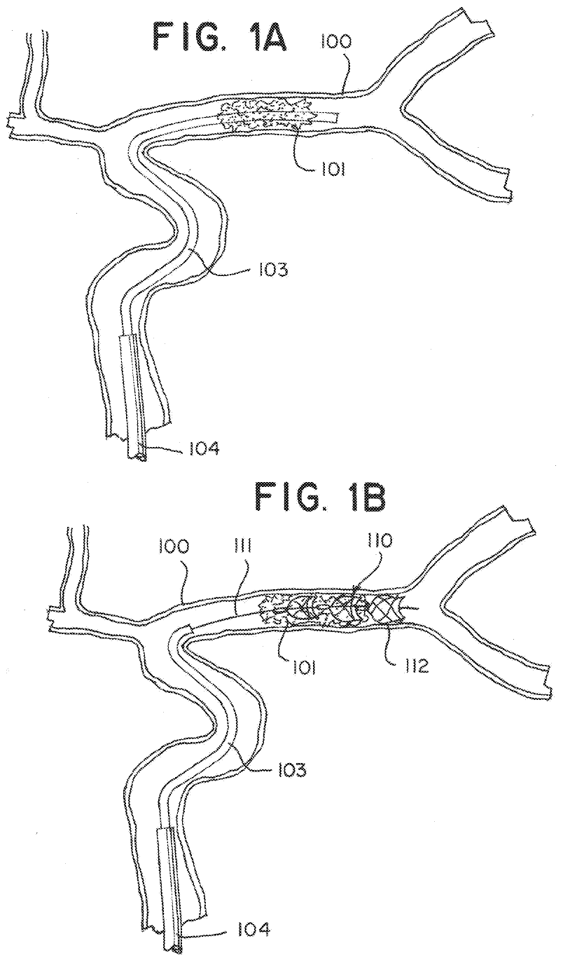

FIGs. 1A and 1B show the delivery of a clot retrieval device 110 for removing a clot 101 from a vessel 100 to a target location. As shown in FIG. 1A, a microcatheter 103 and a guidewire can be inserted into the vasculature 100 via a retrieval catheter 104 and advanced across a clot 101 using conventionally known techniques. When the microcatheter 103 is positioned distal to the clot 101, the guidewire can be removed from the vasculature to allow the clot retrieval device 110 to be advanced through the microcatheter 102. The device 110 can be advanced in a collapsed configuration until the distal tip of the device 110 reaches the distal end of the microcatheter 103.

In FIG. 1B, the microcatheter 103 can be retracted while the position of the device 110 is maintained to deploy the device 110 across the clot 101 in a manner such that the distal end of the device 110 is positioned distally of the clot 101.

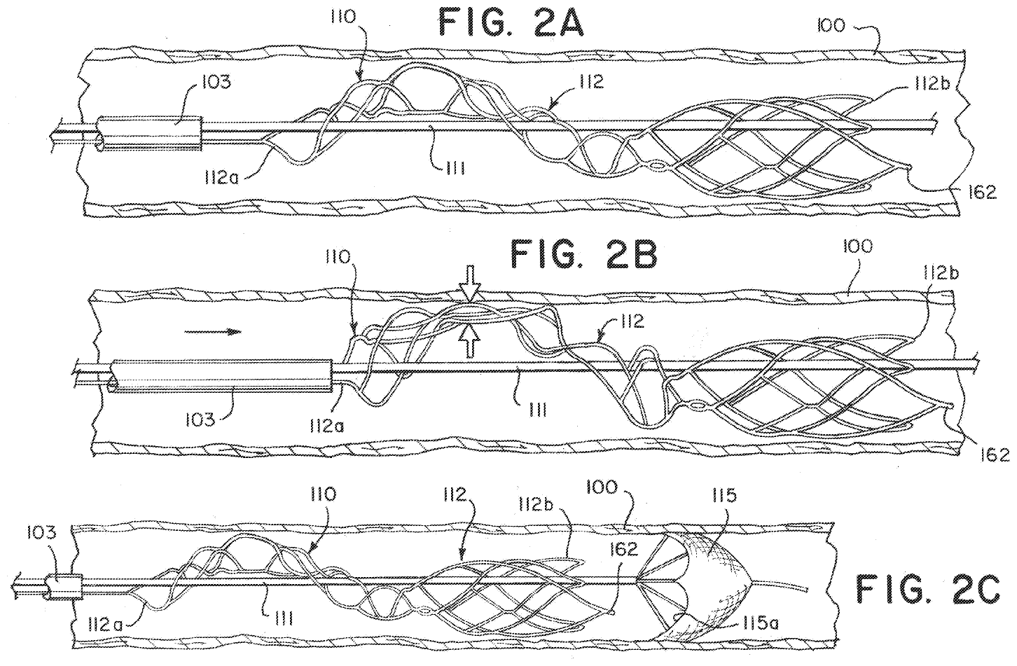

As further illustrated in FIGS. 2A to 2C, the device 110 can include a clot engagement framework 112, a hypotube 111, and a distal emboli protection system 115. The engagement framework 112 can have a collapsed delivery configuration, a clot-engaging deployed configuration, and a clot pinching configuration. The engagement framework can also have a proximal end 112a, a distal end 112b, and a distal tip 162. In the clot pinching configuration, at least a portion of the clot engagement framework 112 can be configured to engage a firm portion of a clot 101 in the deployed configuration and to pinch the clot 101 on movement from the deployed configuration to the clot-pinching configuration. The hypotube 111 can include a proximal end and a distal end at least partially surrounded by the clot engagement framework 112 approximate the hypotube's 111 distal end. The hypotube 111 can also have a clot-gripping outer surface. The distal emboli protection system 115 can be movable from a collapsed delivery configuration within the hypotube 111 to an expanded deployed configuration distal to the hypotube's distal end. The distal emboli protection system 115 can also have a proximal edge 115a.

In FIG. 2A, the device 110 is shown in the deployed configuration. As seen in FIG. 2A, at least a portion of the clot engagement portion 112 encircles the hypotube 111 in a spiral configuration. As shown in FIG. 2B, when a microcatheter 103 is advanced distally into the engagement framework 112, the engagement framework can move to the clot-pinching configuration, which compresses the struts of the proximal end 112a of the engagement framework in order to pinch a clot for extraction from a vessel. This pinching effect can be seen by the illustrated arrows in FIGS. 2A & 2B. The cells in this section are open when the device 110 is fully deployed. As the device 110 is compressed by the microcatheter 103, the cells close “pinching” the clot 101 that is between them for a firmer grip on the clot 101 for removal. As further shown in FIG. 2C, the distal emboli protection system 115 can be advanced through the hypotube 111 in a collapsed delivery configuration and deployed to an expanded configuration outside of the hypotube 111. The emboli protection system 115 can be deployed distally to the distal end of the hypotube 111. The emboli protection system 115 can be deployed distally of the distal tip 162 of the engagement framework 112.

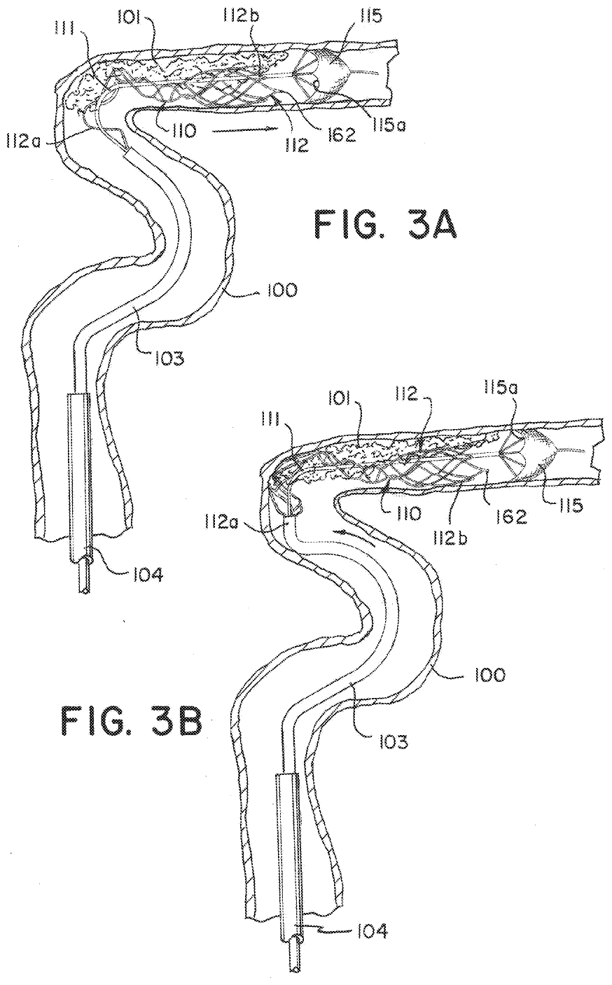

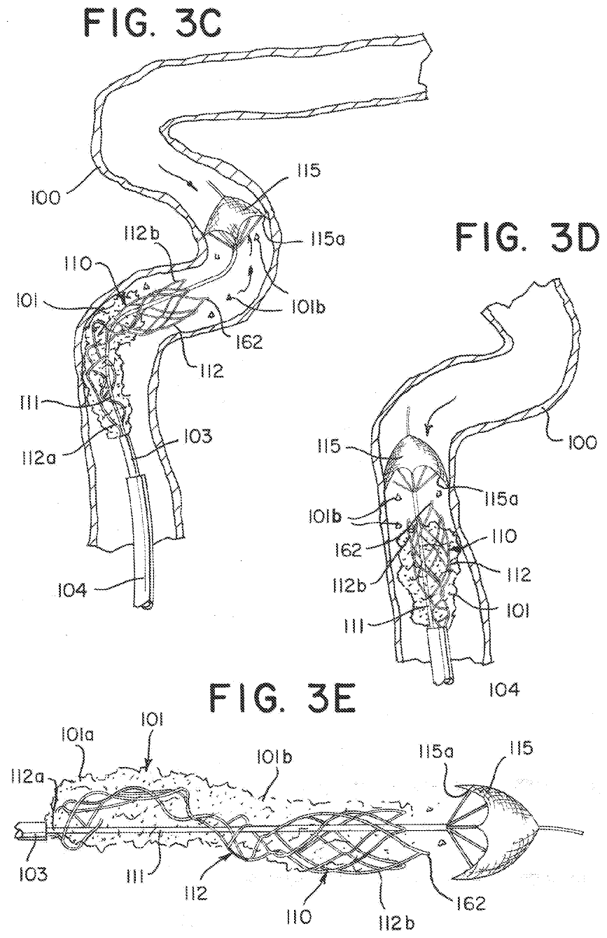

In FIGS. 3A to 3E, removal of a clot 101 from a vessel using a clot retrieval device 110 is depicted. The device 110 can be advanced across a clot 101 as previously described in FIGS. 1A and 1B. As depicted in FIG. 3A, upon retraction of the microcatheter 103, the engagement framework 112 can expand to the deployed configuration. The struts of the engagement framework 112 in the deployed configuration can have a clot gripping surface to engage and hold onto a firm portion of the clot 101a as to aid in removal of the clot 101. After the engagement framework is in the deployed configuration, the distal emboli protection system 115 can be deployed to an expanded configuration distally of the distal tip 162 of the engagement framework 112. Then, as shown in FIG. 3B, the microcatheter 103 can be advanced to pinch a firm portion of the clot between the microcatheter and the engagement framework 112 as previously described. FIG. 3E offers a close-up view of the configuration of the device 110 wherein the engagement framework 112 is in the pinching configuration to pinch a firm proximal end of the clot 101a and the protection system 115 is deployed. After the pinch is achieved, the microcatheter and device can be retracted through the vasculature towards a retrieval catheter 104 as shown in FIG. 3C. During retraction, it is possible that small, softer portions of the clot 101b might break or shear apart from the firmer, larger clot mass 101. The distal emboli protection system 115 can serve as a catch-all mechanism to retain these softer portions of the clot upon removal of the device. The protection system 115 can create a barrier between the soft clot fragments 101b and distal portions of the vessel to prevent the clot fragments 101b from re-entering the vessel and ensuring they are removed with the rest of the clot 101.

In FIG. 3D, the device 110 is removed through the retrieval catheter 104. The distal emboli protection system 115 in the expanded deployed configuration can be sized to travel through a retrieval catheter 104.

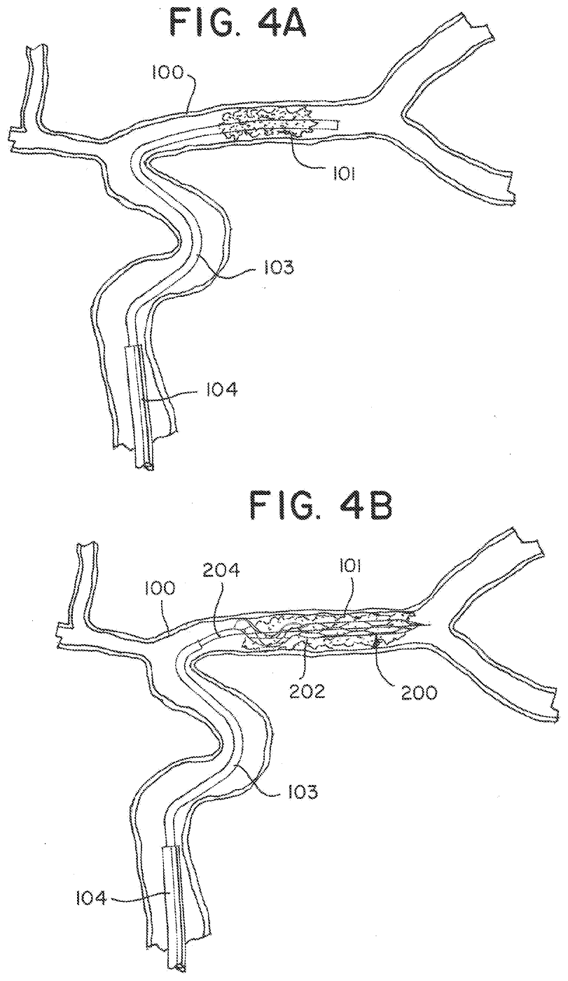

FIGS. 4A and 4B depict the delivery of a clot retrieval device 200 for removing a clot 101 from a vessel 100 to a target location similar to that of FIGs. 1A and 1B. As shown in FIG. 4A, a microcatheter 103 and a guidewire can be inserted into the vasculature 100 via a retrieval catheter 104 and advanced across a clot 101 using conventionally known techniques. The clot 101 can have at least one firm portion and at least one soft portion as previously described. When the microcatheter 103 is positioned distal to the clot 101, the guidewire can be removed from the vasculature to allow the clot retrieval device 200 to be advanced through the microcatheter 103. The device 200 can be advanced in a collapsed configuration until the distal tip of the device 200 reaches the distal end of the microcatheter 103.

In FIG. 4B, the microcatheter 103 can be retracted while the position of the device 200 is maintained to deploy the device 200 across the clot 101 in a manner such that the distal end of the device 200 is positioned distally of the clot 101. The device 200 can include a pull wire 204 and a stentriever 202. The pull wire 204 can be affixed to the stentriever 202 approximate a distal end of the stentriever 202. The stentriever 202 can include a proximal spiral section 206, a distal cylindrical body section 208, and a distal cone 210. At least a portion of the spiral section 206 can encircle the pull wire 204. The stentriever 202 can be configured to expand from a collapsed delivery configuration to a pinching configuration, and then further expand to an expanded configuration. In the collapsed delivery configuration, the stentriever 202 and pull wire 204 can be inside the catheter 103. The device can also include a distal protection system 115 similar to the one described in the preceding figures.

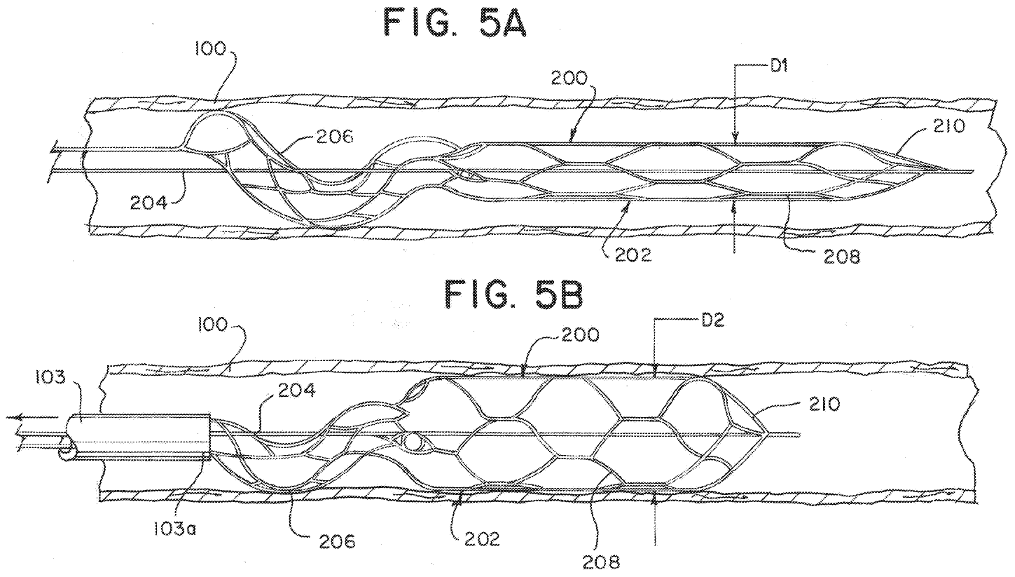

As depicted in FIG. 5A, in the pinching configuration, the stentriever 202 and a portion of the pull wire 204 can be located outside the catheter 103. In the pinching configuration, the distal cylindrical body section 208 can have a first diameter D1. In FIG. 5B, the microcatheter 103 can be advanced distally towards the proximal spiral section 206 to pinch a firm portion of a clot between the microcatheter's distal end 103a and the proximal spiral section 206. When the catheter 103 moves distally, the openings of the proximal spiral portion 206 of the stentriever 202 collapse, pinching the clot, so that the proximal end of the clot is held by the catheter 103 and proximal spiral section 206. As further depicted in FIG. 5B, once the stentriever 202 has exited the microcatheter 103 into the pinching configuration, the pull wire 104 can be retracted relative to the stentriever 202 to expand the stentriever 202 to the expanded configuration. In the expanded configuration, upon movement of the pull wire 204 in relation to the stentriever 202, the distal cylindrical body section 208 can expand to a second diameter D2. In the expanded configuration, the cylindrical body section 208 can engage any soft portions of the clot that might otherwise be vulnerable to shearing or breakage upon removal of the device 200. When the pull wire 204 is retracted proximally in relation to the stentriever 202, the diameter D2 of the cylindrical body section 208 can radially expand to be greater than the diameter D1 of the cylindrical body section 208 in the pinching configuration.

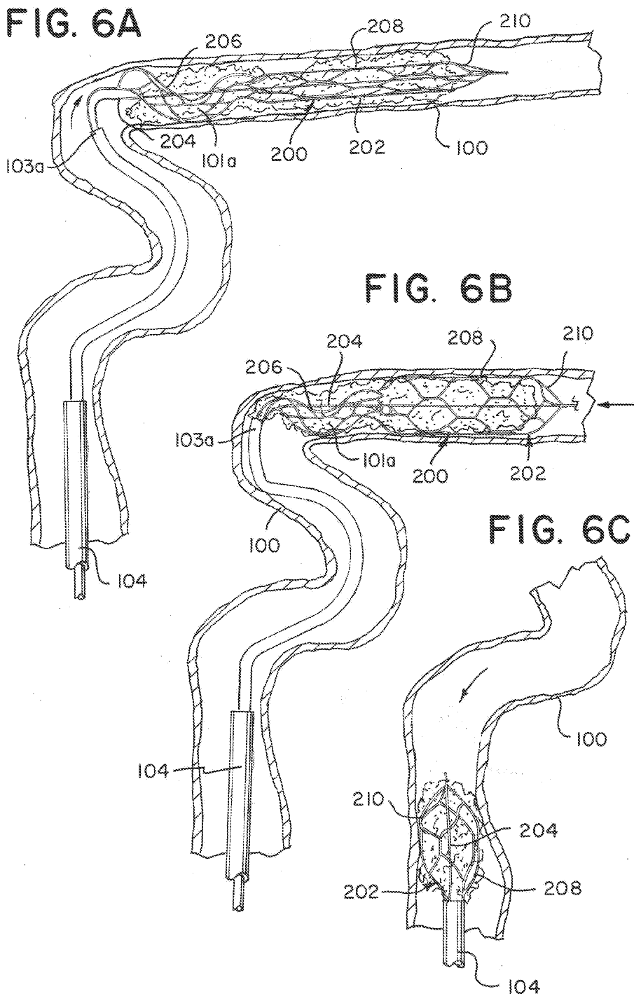

In FIGS. 6A to 6C, removal of a clot 101 from a vessel using a clot retrieval device 200 is depicted. In FIG. 6A, the device 200 can be advanced across a clot 101 as previously described in FIGS. 4A and 4B. As depicted in FIG. 6A, upon retraction of the microcatheter 103, the stentriever 202 can expand to the deployed configuration. The struts of the stentriever 202 in the deployed configuration can have a clot gripping surface to engage and hold onto the clot 101 as to aid in removal of the clot 101. After the stentriever 202 is in the deployed configuration, as shown in FIG. 6B, the stentriever 202 can be expanded to the expanded configuration. Then, the microcatheter 103 can be advanced to pinch a firm portion of the clot 101a between the microcatheter and the proximal spiral section 206 as previously described. After the pinch is achieved, the microcatheter and device 200 can be retracted towards a retrieval catheter 104 as shown in FIG. 6C. During retraction of the device 200, the cylindrical body section 208 can engage any soft portions of the clot 101 that might otherwise be vulnerable to shearing or breakage upon removal of the device 200 to ensure the entire clot 101 is removed.

Other examples can include the distal emboli protection system, as illustrated previously. The distal emboli protection system 115 can serve as a catch-all mechanism to retain these clots upon removal of the device. The protection system 115 can create a barrier between the clot fragments 101b and distal portions of the vessel to prevent the clot fragments 101b from re-entering the vessel and ensuring they are removed with the rest of the clot 101. However, note that this example can capture stray emboli 101b with or without the distal emboli protection system 115.

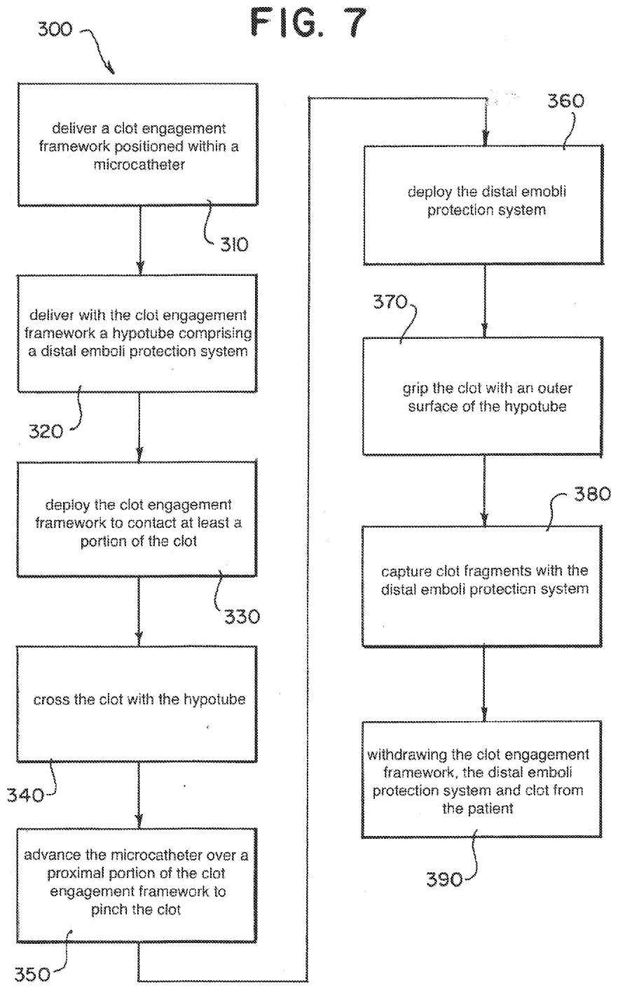

FIG. 7 is a flow diagram depicting a method 300 of treating a patient with an occluded vessel. The method can include delivering a clot engagement framework positioned within a microcatheter (310); delivering with the clot engagement framework a hypotube containing a distal emboli protection system therein to the occluded vessel (320); deploying the clot engagement framework to contact at least a portion of the clot (330); crossing the clot with the hypotube (340); advancing the microcatheter over a proximal portion of the clot engagement framework thereby pinching the clot (350); deploying the distal emboli protection system (360); and withdrawing the clot engagement framework, the distal emboli protection system and clot from the patient. (390)

In the method 300, the clot engagement portion can at least partially encircle the hypotube in a spiral configuration. The method 300 can further include withdrawing the clot engagement framework and the distal emboli protection system into the microcatheter, and removing the clot engagement framework, the distal emboli protection system, and clot from the patient. The method 300 can also include withdrawing the clot, clot engagement framework, and distal emboli protection system in unison. The method 300 can further include gripping the clot with an outer surface of the hypotube (370). The method 300 can also include capturing clot fragments with the distal emboli protection system (380).

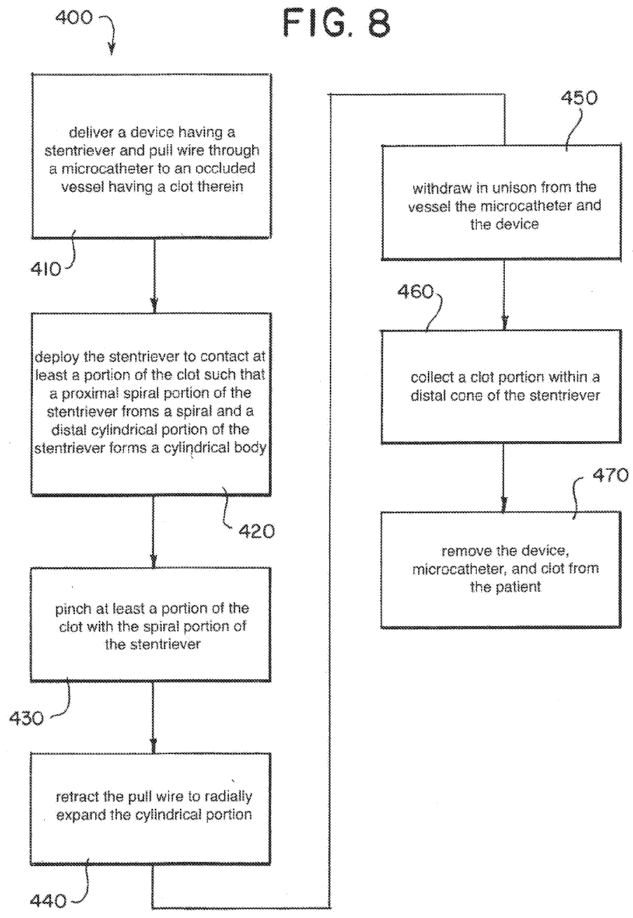

FIG. 8 is a flow diagram depicting a method 400 of treating a patient with an occluded vessel. The occlusion can include a clot. The method 400 can involve delivering a device through a microcatheter (410), the device having a stentriever and pull wire to the occluded vessel; deploying the stentriever to contact at least a portion of the clot such that a proximal spiral portion of the stentriever forms a spiral and a distal cylindrical portion of the stentriever forms a cylindrical body (420); pinching at least a portion of the clot with the spiral portion of the stentriever (430); retracting the pull wire to radially expand the cylindrical portion of the stentriever (440); withdrawing, in unison from the vessel, the microcatheter and the device (450); and removing the device, microcatheter, and clot from the patient (470).

The step 430 of pinching at least a portion of the clot with the spiral portion can further include moving a first portion of the spiral portion proximally towards the microcatheter, thereby causing a second portion of the spiral portion to collapse and pinch the clot. The method 400 can further include collecting at least a portion of the clot within a distal cone of the stentriever (460). The step 460 of collecting at least a portion of the clot within the distal cone can further include retracting the pull wire to radially expand the distal cone. The method 400 can further include positioning the device such that at least a portion of the spiral section encircles the pull wire.

The descriptions contained herein are examples of embodiments of the invention and are not intended in any way to limit the scope of the invention. Modifications apparent to one of ordinary skill in the art following the teachings of this disclosure are intended to be within the scope of the claims which follow.

1. A clot removal device for removing a clot from a body vessel, the clot removal device comprising:
a clot engagement framework comprising a collapsed delivery configuration, a clot-engaging deployed configuration, and a clot-pinching configuration such that at least a portion of the clot engagement framework is configured to engage a clot in the deployed configuration and to pinch a clot on movement from the deployed configuration to the clot-pinching configuration;
a hypotube comprising a proximal end and a distal end at least partially surrounded by the clot engagement framework approximate the hypotube's distal end; and
a distal emboli protection system movable from a collapsed delivery configuration within the hypotube to an expanded deployed configuration distal to the hypotube's distal end;
wherein at least a portion of the proximal spiral section encircles the hypotube.

2. The device of claim 1 wherein the hypotube further comprises a clot-gripping outer surface.

3. The device of claim 1 wherein at least a portion of the clot engagement portion encircles the hypotube in a spiral configuration.

4. The device of claim 1 wherein the hypotube is configured to cross the clot.

5. The device of claim 1 wherein the distal emboli protection system in the expanded deployed configuration is sized to travel through a retrieval catheter.

6. A method of treating a patient with an occluded vessel, the occlusion comprising a clot and the method comprising the steps of:
delivering a clot engagement framework positioned within a microcatheter;
delivering with the clot engagement framework a hypotube comprising a distal emboli protection system therein to the occluded vessel;
deploying the clot engagement framework to contact at least a portion of the clot;
crossing the clot with the hypotube;
advancing the microcatheter over a proximal portion of the clot engagement framework, thereby pinching the clot;
deploying the distal emboli protection system; and
withdrawing the clot engagement framework, the distal emboli protection system and clot from the patient;
wherein at least a portion of the proximal portion of the clot engagement framework encircles the hypotube.

7. The method of claim 6 further comprising:
withdrawing the clot engagement framework, and the distal emboli protection system into the microcatheter; and
removing the clot engagement framework, the distal emboli protection system, and clot from the patient.

8. The method of claim 6 further comprising:
withdrawing the clot, clot engagement framework, and distal emboli protection system in unison.

9. The method of claim 6 further comprising:
gripping the clot with an outer surface of the hypotube.

10. The method of claim 6, further comprising:
capturing clot fragments with the distal emboli protection system.

11. The method of claim 6 wherein the clot engagement portion at least partially encircles the hypotube in a spiral configuration.

12. A method of treating a patient with an occluded vessel, the occlusion comprising a clot and the method comprising the steps of:
delivering a device comprising a stentriever and pull wire to the occluded vessel through a microcatheter;
deploying the stentriever to contact at least a portion of the clot such that a proximal spiral portion of the stentriever forms a spiral and a distal cylindrical portion of the stentriever forms a cylindrical body;
pinching at least a portion of the clot with the spiral portion of the stentriever;
retracting the pull wire to radially expand the cylindrical portion of the stentriever;
withdrawing in unison from the vessel the microcatheter and the device; and
removing the device, microcatheter, and clot from the patient.

13. The method of claim 12 wherein the step of pinching at least a portion of the clot with the spiral portion further comprises moving a first portion of the spiral portion proximally towards the microcatheter, thereby causing a second portion of the spiral portion to collapse and pinch the clot.

14. The method of claim 12 further comprising collecting at least a portion of the clot within a distal cone of the stentriever.

15. The method of claim 14, further comprising:
retracting the pull wire to radially expand the distal cone.

16. The method of claim 12, further comprising:
positioning the device such that at least a portion of the spiral section portion the pull wire.
